(19) United States
(12) Patent Application Publication
(10) Pub. No.: US 2011/0287380 A1
(43) Pub. Date: Nov. 24, 2011

(54) CLINICAL ROOT CANAL THERAPY INSTRUMENT EQUIPPED WITH ILLUMINATION

(76) Inventor: Yan-Zhang LIN, Nantou County (TW)

(21) Appl. No.: 12/783,289

(22) Filed: May 19, 2010

(51) Int. Cl. A61B 1/247; A61B 1/24

(52) U.S. Cl. 433/30; 433/29

(57) ABSTRACT

A clinical root canal therapy instrument equipped with illumination comprises a handgrip and a therapy element. The handgrip includes a hollow lighting section which has a coupling portion fastened to the therapy element. The therapy element can be a root canal oral mirror. The lighting section holds at least one lighting element connected to a power source and includes at least one aperture on the wall thereof to emit light. The therapy element has one end formed a pushing portion to isolate a patient's tongue or cheek. The handgrip and the root canal oral mirror form an obtuse angle therebetween. The pushing portion includes at least one light penetrating element on an outer wall thereof. Thus the instrument of the invention can function as an independent flashlight or be fastened to the root canal oral mirror to provide illumination to aid a dentist to inspect or treat the patient's teeth.

### FIELD OF THE INVENTION

The present invention relates to an oral mirror used in dental clinic and particularly to a dental instrument equipped with illumination to aid dentists to perform inspection or treatment for patient's teeth.

### BACKGROUND OF THE INVENTION

A conventional oral mirror used in dental clinic, referring to FIG. 1, does not provide illumination. It merely aims to push aside a patient's tongue or cheek to expose teeth to allow a dentist to observe the teeth. To perform a thorough inspection or treatment for the teeth in the oral cavity, the dentist usually has to rely on a flashlight to project to the teeth where therapy is needed. During treatment, the dentist has to hold a therapy instrument with one hand and the oral mirror with another hand, so that holding the flashlight becomes impossible. Thus the dentist commonly has to wear a head lamp and bend the body to project light from the head lamp to the interior of patient's oral cavity, or rely on an external lighting fixture to provide sufficient illumination for inspection and therapy. It is awkward and troublesome, and still has room for improvement.

### SUMMARY OF THE INVENTION

Therefore, it is an object of the invention to provide a clinical root canal therapy instrument equipped with illumination that can function as an independent flashlight and also can be coupled with an oral mirror to provide illumination when a dentist performs treatment for patient's teeth, and also to solve the problem of the conventional technique that requires the dentist to hold a root canal therapy instrument with one hand and a treatment instrument with another hand and cannot hold the flashlight to illuminate the interior of a patient's oral cavity.

To achieve the foregoing object, the invention provides a clinical root canal therapy instrument equipped with illumination that includes a handgrip and a therapy element which can be a root canal oral mirror. The handgrip has a hollow lighting section and a coupling portion at a front side of the lighting section. The lighting section holds at least one lighting element connected to a power source to generate light and has at least one aperture formed on the wall thereof to emit light from the lighting element. The coupling portion is fastened to one end of the root canal oral mirror which has another end formed a pushing portion to isolate a patient's tongue or cheek.

The handgrip can function as an independent flashlight and also can be coupled to the root canal oral mirror. Light generated by the lighting element can be emitted through the aperture to aid the dentist in inspecting or treating the patient's teeth and overcome the problem of holding a root canal therapy instrument with one hand and a treatment instrument with another hand and cannot hold the flashlight to illuminate the interior of the patient's oral cavity.

The foregoing, as well as additional objects, features and advantages of the invention will be more readily apparent from the following detailed description, which proceeds with reference to the accompanying embodiments and drawings. The embodiments serve merely for illustrative purpose and are not the limitations of the invention.

### BRIEF DESCRIPTION OF THE DRAWINGS

FIG. 1 is a perspective view of a conventional oral mirror used in dental clinic.

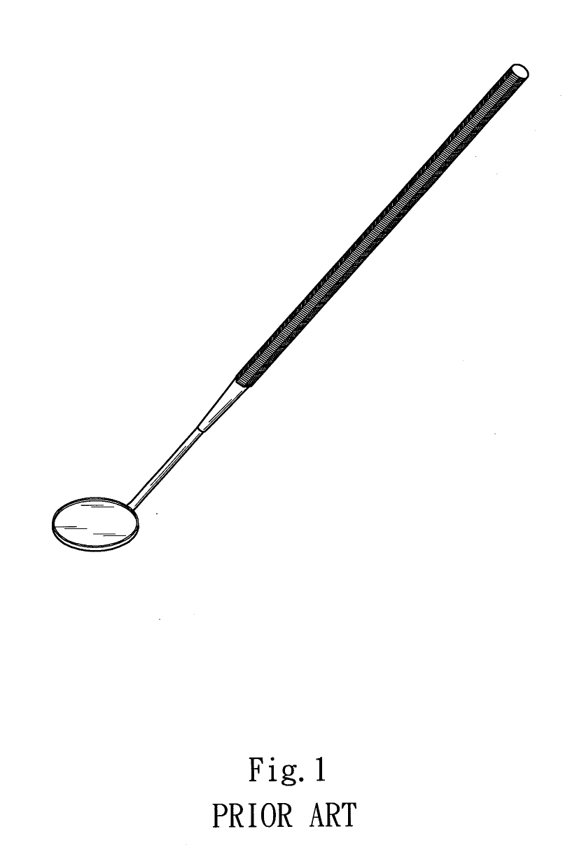

FIG. 2 is a plane schematic view of an embodiment of a handgrip of the invention.

FIG. 3 is a schematic view of an embodiment of the handgrip coupled to a root canal oral mirror according to the invention.

FIG. 4 is a schematic view of an embodiment of the handgrip coupled to the root canal oral mirror equipped with a light penetrating element according to the invention.

### DETAILED DESCRIPTION OF THE PREFERRED EMBODIMENTS

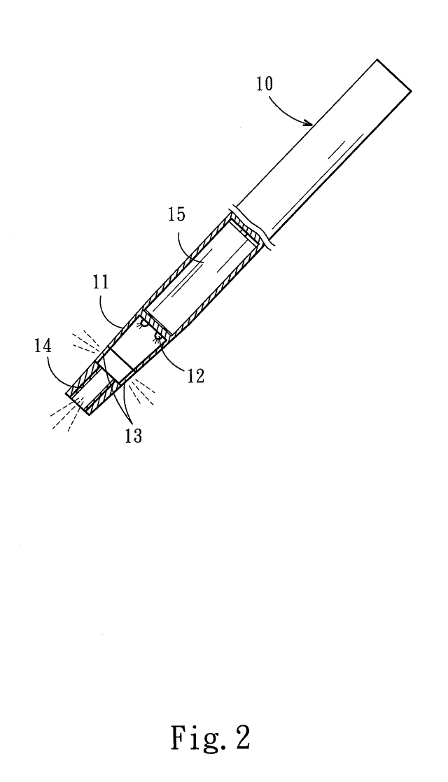

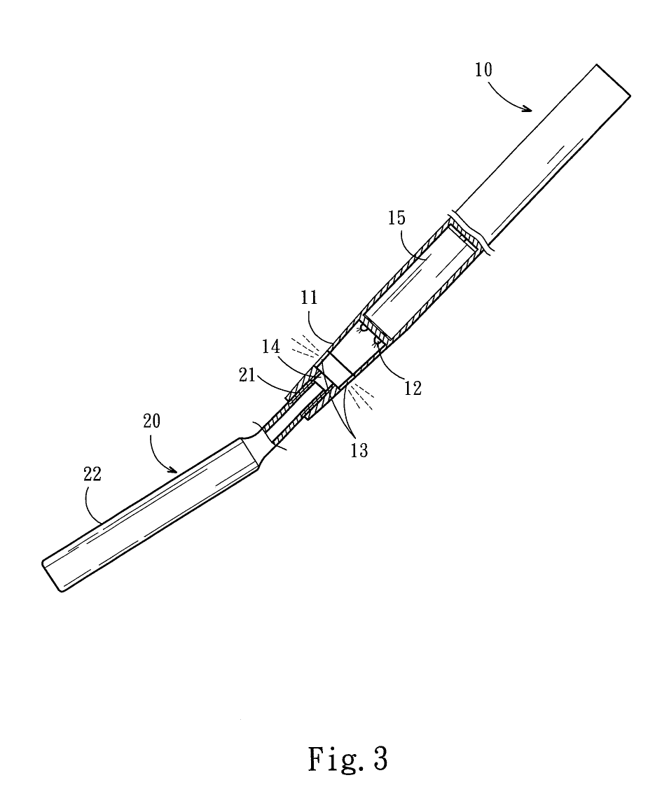

Please refer to FIGS. 2 and 3, the invention provides a root canal therapy instrument equipped with illumination which includes a handgrip 10 and a therapy element. The handgrip 10 has a hollow lighting section 11 to hold at least one lighting element 12 inside connected to at least one power source 15 to generate light. The lighting section 11 also has at least one aperture 13 on the wall thereof to emit light generated by the lighting element 12. The lighting section 11 has a coupling portion at the front side that is a screw hole 14 in this embodiment.

The therapy element has one end coupled to the coupling portion. In this embodiment, the therapy element is a root canal oral mirror 20 which form an obtuse angle with the handgrip 10. The root canal oral mirror 20 has a hollow screw 21 at one end to fasten to the screw hole 14 of the lighting section 11, and another end formed a pushing portion 22 to isolate a patient's tongue or cheek.

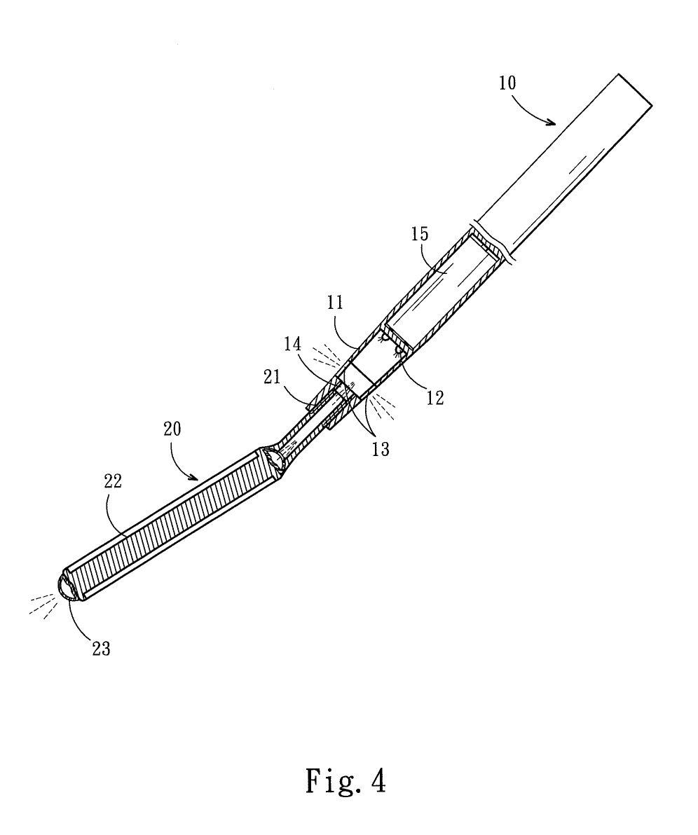

Refer to FIG. 4 for another embodiment of the invention. The pushing portion 22 has at least one light penetrating element 23 on an outer wall thereof. In this embodiment, the light penetrating element 23 is hollow and made of an elastic and pliable transparent material to surround the pushing portion 22. The light penetrating element 23 also has a hole communicating with the screw 21 to allow the light of the lighting element 12 to enter into the screw 21 and then the light penetrating element 23, so that the light is emitted from the light penetrating element 23.

By means of the structure set forth above, the lighting element 11 of the handgrip 10 can generate light and the light can be emitted outside through the aperture 13 and screw hole 14 to serve as an independent flashlight.

When the handgrip 10 and the root canal oral mirror 20 are fastened together, the root canal oral mirror 20 can be disposed into a patient's oral cavity to push away the patient's tongue or cheek to facilitate the dentist's inspection or treatment for the patient's teeth. The lighting section 11 is close to the pushing portion 22, thus can illuminate the interior of the oral cavity to aid the dentist to inspect or treat the patient's teeth. It also can overcome the problem encountered by the dentist in the conventional technique of holding a root canal therapy instrument with one hand and a treatment instrument with another hand and cannot hold the flashlight to illuminate the interior of the patient's oral cavity.

What is claimed is:

1. A clinical root canal therapy instrument equipped with illumination, comprising a handgrip and a therapy element, the handgrip including a hollow lighting section which includes a coupling portion fastened to the therapy element, the lighting section including at least one lighting element inside connected to at least one power source and at least one aperture on the wall thereof to emit light.

2. The clinical root canal therapy instrument of claim 1, wherein the therapy element is a root canal oral mirror which includes one end fastened to the coupling portion and another end formed a pushing portion to isolate a patient's tongue or cheek.

3. The clinical root canal therapy instrument of claim 2, wherein the coupling portion is a screw hole and the one end of the root canal oral mirror is a hollow screw.

4. The clinical root canal therapy instrument of claim 2, wherein the root canal oral mirror and the handgrip form an obtuse angle therebetween.

5. The clinical root canal therapy instrument of claim 2, wherein the pushing portion includes at least one light penetrating element on an outer wall thereof that is made of an elastic and pliable transparent material and includes a hole communicating with the screw.

6. The clinical root canal therapy instrument of claim 5, wherein the light penetrating element surrounds the pushing portion.
